(19) United States
(12) Patent Application Publication
(10) Pub. No.: US 2024/0044868 A1
(43) Pub. Date: Feb. 8, 2024

(54) CONTROLLABLY EXPOSED CHAMBER FOR BIOLOGICAL SPECIMEN

(71) Applicant: Analog Devices, Inc., Wilmington, MA (US)

(72) Inventors: Max Marcel Shulaker, Cambridge, MA (US); Samuel H. Fuller, Wellesley, MA (US)

(21) Appl. No.: 18/229,618

(22) Filed: Aug. 2, 2023

Related U.S. Application Data
(60) Provisional application No. 63/370,225, filed on Aug. 2, 2022.

(51) Int. Cl. G01N 33/497

(52) U.S. Cl. CPC G01N 33/497

(57) ABSTRACT

A system for gas sensing of a biological specimen can include a chamber, such as to receive the biological specimen therewithin. The system can also include a gas sensing unit to be coupled to the chamber and separated therefrom by a moveable separator. A gas sensor included in the gas sensing unit can be selectively exposed to a fluid headspace of the first chamber, such as a gas environment associated with the biological specimen, upon modification or moving of a separator.

### CLAIM OF PRIORITY

This application claims priority to U.S. Provisional Ser. No. 63/370,225, filed Aug. 2, 2022, which is hereby incorporated by reference herein in its entirety.

### BACKGROUND

Blood cultures are blood samples submitted to microbiology laboratory for the purpose of identifying the organism(s) in the blood that can cause a bacterial, fungal, or other infection. Blood culture results can be utilized to determine an appropriate course of antibiotic treatment and/or a source of infection and can assist in the exclusion of sepsis and other infections. Blood culture results can also assist in identifying specific pathogens to help treat patients with a more specific infection. The process of obtaining a blood culture can involve drawing a blood sample and encapsulating the blood sample in a vial for transport to a microbiology laboratory for analysis of the blood sample.

Blood culture vials can include a sample collection fluid and a nutrient medium that has been mixed with a blood sample that is stored in the blood culture vial. The vial containing the sample with blood can be stored at a specific temperature until it arrives at a microbiology laboratory, where the vial is examined to determine whether an organism or organisms are present in the blood sample. If the culture reveals that microorganisms, such as bacteria, are present in the blood sample, then other tests can be performed to determine additional detailed information about the microorganism and how to best treat the patient.

Microorganisms, such as bacteria or fungus, present in a blood sample or other samples can be identified based upon the types of organisms they are. By using a particular culture test, such as a blood culture, a patient can receive a specific type of treatment depending on the bacterial strain found in the blood sample. A blood culture bottle containing a blood sample can be transported to a microbiology laboratory, where an organism can be grown and identified from the blood sample.

Similar methods can be used to identify cultures in other biological specimens, such as cerebrospinal fluid, pleural fluid, and spinal fluid, as well as cultures of other body fluids such as urine, sputum, throat swabs, eye swabs, wounds, body fluids or secretions, and so forth.

### SUMMARY

A biological specimen can be contained within a vial to be tested, for instance for specific cultures or other types of biochemical or biological tests. Such testing can generally be performed using a manual procedure requiring several hours to weeks, during which time the culture vial is not available for other use (e.g., transferring a sample of a positive blood culture bottle to a petri dish for additional culturing and microorganism isolation). To do so, a technician often must transfer the content from the original container to a different vessel, where subsequent tests can be performed (such as incubating the sample for isolation and identification, or performing antibiotic susceptibility testing). The process of transferring culture vial contents to another vessel is labor intensive and can have numerous associated risks of contamination, both to the technician as well as to the biological sample. In addition, after the culture vial contents have been transferred to the different vessel, they might be read using a manual, visual procedure. This can introduce a large number of sources of error, including a technician having to incorrectly read the vial, the technician having to interpret an incorrectly labeled vial, or the technician having to read a non-optimal location on the vial that does not correspond to a positive growth site on the vial.

In another approach, a gas sensor or array of gas sensors can be used to detect a gas produced by the bacteria. The vial can receive the biological specimen. The vial can be attached to the gas sensor for detecting the presence of bacteria in the culture fluid. If the gas sensor array detects the presence of bacteria, then a technician similarly needs to perform the time-consuming transfer of some of the contents of the culture vial to a separate vessel, which requires manual disassembly from the gas sensor apparatus in order to access the contents of the vial. The present inventors have recognized a need for techniques for rapid gas sensing of a biological specimen within a container and obviating the need for some manual operations required by other approaches. For example, a gas sensor can be fluidly separated from a biological specimen. The gas sensor can be selectively exposed to a gas environment associated with the biological specimen.

This document describes a gas sensing system including a liquid-protected gas sensor. Such a system can include or use a first chamber to receive the biological specimen therewithin and a second chamber carrying the gas sensor. For example, the gas sensor can be isolated from the first chamber by an openable seal to transition from a first state into a second state upon opening the seal to allow gas from the first chamber to diffuse or otherwise flow into the second chamber for sensing by the gas sensor. The second chamber can be isolated from an ambient environment in at least the second state. The gas sensor can include or use a field effect transistor (FET) such as a carbon nanotube FET (CNFET). Also, the gas sensor can include or use a camera, a spectroscopic imaging sensor, a hyperspectral imaging sensor, a colorimetric imaging sensor, a fluorescence response imaging sensor, or a chromatic sensor.

The openable seal can be closed in the first state with a positive second pressure in the second chamber relative to a first pressure in the first chamber. Also, the seal can include a first component that can be movable with respect to a second component to provide at least one opening in the seal to transition from the first state into the second state. The openable seal can be included in a set with a second seal that can be exchanged with a first seal. For example, the system can include a seal perforator actuatable to break the openable seal for transition from the first state to the second state. The openable seal can include a gas-permeable membrane included such as to impede liquid flow therethrough. The openable seal can include a closable port of the first chamber. Gas travel through the closable port can be physically impeded in the first state and gas travel through the closable port can be provided in the second state. In an example, the first chamber can be included as a part of a second chamber plug. Here, the second chamber plug can be sized and shaped to interface with the second chamber to fluidly enclose the second chamber by plugging an opening of the second chamber.

Each of the non-limiting examples described herein can stand on its own, or can be combined in various permutations or combinations with one or more of the other examples.

This Summary is intended to provide an overview of the subject matter of the present patent application. It is not intended to provide an exclusive or exhaustive explanation of the invention. The detailed description is included to provide further information.

### BRIEF DESCRIPTION OF THE FIGURES

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1A depicts an example of a fluid transfer system.

FIG. 1B shows a perspective view of a device for facilitating fluid transfer.

FIG. 1C shows a side view of a device for facilitating fluid transfer.

FIG. 2 depicts an example of a gas sensing system.

FIG. 3 depicts another example of a gas sensing system.

FIG. 4A depicts an example of a gas sensing system in a first position.

FIG. 4B depicts an example of a gas sensing system in a first position.

FIG. 5 is a flowchart that describes a method of using a gas sensing system.

### DETAILED DESCRIPTION

A biological specimen, such as a human blood sample, can be received within a vial to be tested for culture. One approach to testing for cultures involves placing several different specimens within individual containers, such as vials. A technician then performs multiple manual operations, such as opening a container, adding reagents, mixing the specimens, and incubating the samples. Following incubation, a technician can visually assess an individual vial for a presence of a target culture to determine a testing result. This can introduce many sources of error, including a technician having to incorrectly read the vial, the technician having to interpret an incorrectly labeled vial, or the technician having to read a non-optimal location on the vial that does not correspond to a positive growth site on the vial. Another challenge with this approach is that the result is generally not ascertainable for several days or weeks following retrieval of the biological specimen from a human patient. Further, performing such multiple manual operations can potentially lead to a loss of specimen, reagent waste, cross-contamination, and other errors in the preparation of the specimen.

In another approach to testing of a biological specimen, a gas sensor can be used to inspect the biological specimen. Here, a vial can receive the biological specimen and one end of the vial can be sealed shut. The other end of the vial can be attached to the gas sensor, which can be placed into (or in fluid communication with) the culture fluid. The gas sensor can detect when the culture fluid changes, and thus, when bacteria have been detected in the culture fluid. A challenge with this approach is that the gas sensor can be damaged by the biological specimen. For example, a liquid biological specimen can degrade a gas sensor by causing erosion, dilution, electrical shorting, or clogging of the gas sensor. Also, this approach may involve similar manual operations as described above, such as transfer of the culture vial to a separate vessel and involves a technician removing the vial from the sensor when it detects a change in the culture fluid.

The present inventors have recognized techniques for handling and inspection of a biological specimen within a container and obviating the need for certain manual operations of other approaches. For example, a gas sensor can be fluidly separated from a biological specimen contained within a first chamber. The gas sensor can be selectively exposed to a fluid headspace of the first chamber, such as a gas environment associated with the biological specimen, upon modification or moving of a separator such as a seal. For example, the gas sensor can be located within a plug or cap of a container including the first chamber. The act of coupling the plug or cap with the container can be used to manipulate the separator such that the gas sensor is exposed to the gas headspace of the first chamber. Also, the gas sensor can be located within a second chamber, and the first and second chamber can be arranged within the same container. Here, the first chamber and the second chamber can be selectively fluidly connected upon modification or moving of the separator, such as to expose a gas environment associated with a liquid biological specimen to the gas sensor.

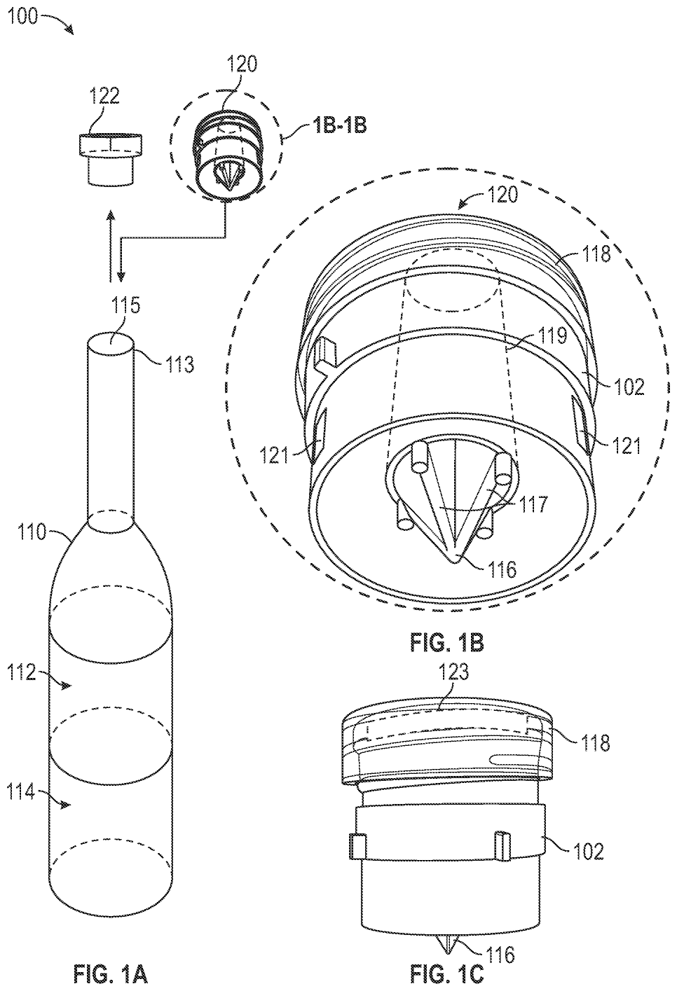

FIG. 1A depicts an example of a fluid transfer system. A fluid transfer system 100 can include a specimen container 110, such as a vial, for receiving and retaining a biological specimen 114 therewithin. The fluid transfer system 100 can involve selectively exposing the biological specimen 114 with a sensor or a sensing system, e.g., to monitor for growth or condition of the biological specimen 114, while ensuring to specimen container 110 remains user-sealable and reducing contamination during culturing of contents within the specimen container 110.

The specimen container 110 can include an openable, fluid-impermeable seal 115 at a sealed aperture 113 of the specimen container. For example, the fluid-impermeable seal 115 can include a septum including, e.g., silicone, rubber, or plastic affixed (e.g., adhered or fastened) to the specimen container 110. The seal 115 can provide a sterile environment within the specimen container 110, for example, by preventing contamination or inadvertent ingress of fluid, dust particles, or other bacteria into the specimen container 110. In an example, the seal 115 can include a gas-permeable membrane, such as such as a polyethylene, polytetrafluoroethylene, or other type of thin film or membrane material. The gas-permeable membrane can include a liquid impervious property, such as to impede liquid flow therethrough.

Generally, a compliant e.g., resilient membrane of the seal 115 can define a passageway to an interior of the specimen container 110 upon application of a force to the membrane (e.g., a piercing or puncturing force). For example, a syringe needle (or some other piercing element) can be used to puncture the membrane. Upon removal of the syringe from the membrane, the membrane can return to its resting, sealed, or unperforated state (e.g., resealed). The resealing can prevent leakage or escaping of the biological specimen 114 from the specimen container 110. Such biological specimen 114 can include one or more of liquid, solid, colloidal, and gaseous components. The biological specimen 114 can include one or more liquids such as blood, plasma, cerebrospinal fluid, synovial fluid, urine, sweat, saliva, transcutaneously obtained fluids (TTF), sputum, mucus, stool, gastric contents, and tissue. While the fluid-impermeable seal 115 can facilitate certain fluid-transfer techniques, e.g., by withdrawal or depositing through a need or cannula, a seal 115 in an unopened, resealed, or partially sealing state can present challenges to culturing and sensing the biological specimen 114.

In an example, the fluid transfer system 100 can include a device 120 for providing access to the specimen container 110. In an example, the device 120 can irreversibly sever and unseal the fluid-impermeable seal 115, such as to facilitate fluid transfer between the specimen container 110 and a culturing or sensing system, e.g., located external to the specimen container 110. For example, certain culturing or sensing systems can involve fluid transfer, such as diffusion of a gas headspace, at a flow rate that is unfeasible to provide via a needle or cannula inserted through the seal 115, e.g., due to a constriction through a lumen of the needle or cannula. Thus, the device 120 can assist in providing desired fluid access to the specimen container 110, by irreversibly severing and unsealing the fluid-impermeable seal 115 of the specimen container, while remaining user resealable. Such a configuration can be helpful in adapting certain specialized equipment, e.g., a blood vial including a resealing septum configured for access via a syringe, to certain advanced detection and culturing systems that could otherwise be incompatible or undesirable for use with an in-tact seal or septum of a container.

FIG. 1B and FIG. 1C show detailed views of the device 120 of FIG. 1A. The device 120 can include a fitting 102 sized and shaped to be coupled to the aperture 113 of the specimen container 110 (as depicted in FIG. 1A). As described herein, the fitting 102 can define a body of the device 120, and such a body can press tightly against the aperture 113 of the specimen container 110 to plug, fill, or jam the aperture 113. For example, the fitting 102 can include a viscous material such as an elastomer arranged to restrict uncoupling of the fitting 102 from the specimen container 110. Also, the fitting 102 can include a taper, e.g., a conical or stepped conical tip, for insertion into the aperture 113. The fitting 102 can also include one or more retaining elements for holding the fitting 102 to the specimen container 110 and for maintaining a fluid-tight seal between the fitting 102 and the aperture 113.

In an example, the fitting 102 can be at least partially inserted into the aperture 113. Coupling of the fitting 102 into the aperture 113 can establish a lasting, semipermanent, or permanent junction. For example, the junction can require a force greater than 5 newtons (N) to break or unplug. Coupling of the fitting 102 into the aperture 113 can also involve irreversibly severing and unsealing the fluid-impermeable seal 115. Here, the fitting 102 can include or be attached to a perforator 116, e.g., at or near an end of the fitting 102. The perforator 116 can be arranged to irreversibly sever and unseal the seal 115, e.g., during insertion of the fitting 102 into the aperture. For example, the perforator 116 can include a seal dilator 117. The seal dilator 117 can include ribs, fins, or concentric ridges for dilating the seal 115. The seal dilator 117 that can be sized and shaped such that upon the perforator 116 being thrust toward the seal 115, the dilator 117 displaces greater than 20% of a surface area of the seal 115, such as creating an opening of the seal having a cross-sectional area of at least 20% of the surface area of the seal 115. In an example, the dilator 117 can displace greater than 50% of the surface area of the seal 115 such as creating an opening of the seal having a cross-sectional area of at least 50% of the surface area of the seal 115. In an example, a pressure-actuated mechanism, such as a linear actuator or solenoid, can be operatively connected to the perforator 116 to move the perforator 116 to break the seal 115. Such a pressure-actuated mechanism can be actuated upon being exposed to fluid pressure greater than a predetermined threshold level. In an example, the perforator 116 can be spring-biased to break the seal 115. The perforator 116 can be configured to be actuated automatically, such as via a timer-based or controller-based system.

The fitting 102 can include a lumen 119 extending between first and second ends of the fitting. The lumen 119 can extend toward the perforator 116 such that upon severing and unsealing of the seal 115 via coupling of the fitting 102 to the aperture 113, a first end of the lumen 119 becomes fluidly connected with a headspace 112 of the specimen container 110. For example, the lumen 119 can establish an access port to the biological specimen 114 after the seal 115 has been severed and unsealed by the perforator 116. In an example, the lumen 119 can include a cross-sectional diameter greater than about 2.5 millimeters (mm). For example, within a range of about 2.5 mm and about 2.5 centimeters (cm), or within a range of about 3 mm and about 2.5 centimeters. For example, the cross-sectional diameter can be significantly greater than a diameter of a conventional needle, e.g., greater than about 2 millimeters (mm).

The device 120 can also include a user-replaceable sealing enclosure 118. For example, the sealing enclosure 118 can be sized and shaped to house at least one biological specimen growth indicator 123 at a second end of the lumen 119. For example, the at least one biological specimen growth indicator 123 arranged to be enclosed by the sealing enclosure 118 separately from an ambient environment external to the specimen container. The sealing enclosure 118 can be selectively user-engageable to confine or capture fluid escaping from the second end of the lumen 119 of the fitting, e.g., after the seal has been unsealed by the perforator 116. Thus, the sealing enclosure 118 can define a chamber or cavity for receiving a portion of the biological specimen 114. In an example, the user-replaceable sealing enclosure 118 can include a removable cap included such as to removably couple with a coupling feature of the fitting 102. Here, the coupling feature can include threading on the fitting 102 or the removable cap, and the sealing enclosure 118 can be user-engageable to confine fluid escaping from the second end of the lumen 119 via tightening the removable cap onto the fitting 102. The coupling feature can also include, e.g., other forms of engaging components, such as a clam-shell fitting, snap fit, bayonet fitting, twist fitting, or a combination thereof. In an example, at least one of the fitting 102 or the sealing enclosure 118 can be formed of a translucent or transparent material to allow imaging into the specimen container while the sealing enclosure 118 remains engaged.

In an example, the at least one biological specimen growth indicator 123 can include a biological culture media, e.g., nutrient agar. For example, the culture media can be a tab, paddle, or a mini plate of nutrient agar housed by the sealing enclosure 118 and arranged to be contacted by fluid from the specimen container via liquid travel from the specimen container 110 through the lumen 119. Here, following coupling of the device 120 with the specimen container 110, the specimen container 110 can be inverted such as to allow liquid to travel from the specimen container 110, through the lumen 119, and to contact the biological culture media of the at least one biological specimen growth indicator 123. Such an approach to cell culturing using, e.g., septum-sealed containers can enable monitoring of cell cultures even in situations or environments where liquid-culture monitoring techniques are unavailable or unfeasible. Thus, a biological specimen growth indicator 123 including biological culture media can be helpful in adapting certain specialized equipment, e.g., a blood vial including a resealing septum configured for access via a syringe, to accommodate circumstances where limited monitoring equipment is available.

In an example, the at least one biological specimen growth indicator 123 can include a gas sensor. The gas sensor can include, e.g., a semiconductor-based gas sensor, such as a field effect transistor (FET), e.g., a carbon nanotube FET (CNFET), silicon carbide FET (SiCFET), metal-oxide-semiconductor FET (MOSFET), graphene FET (GFET), transition metal dichalcogenide FET (TMDFET), or black phosphorus FET (BPFET). Also, the gas sensor can include a polymer-based gas sensor, an optical gas sensor, a chromatic gas sensor, an atomic absorption spectrometer, a catalytic gas sensor, a catalytic combustion gas sensor, an electrochemical sensor, a resistive gas sensor, or the like. The at least one biological specimen growth indicator 123 can also include or use a camera, a spectroscopic imaging sensor, a hyperspectral imaging sensor, a colorimetric imaging sensor, or a fluorescence response imaging sensor. The gas sensor can also include at least one light emitting illumination element and at least one light receiving photodetector or other element such that the gas sensor 125 can function as a photoelectrochemical sensor.

In an example, the at least one biological specimen growth indicator 123 can include an internal fluid sensor for measuring at least one fluid condition within at least one of the lumen 119 or the specimen container 110. In an example, the device 120 can also include electrical contacts 121 located external to the specimen container 110 and, e.g., electrically coupled to the internal fluid sensor. In an example, the electrical contacts 121 can communicate internal fluid sensor data to an external location while the sealing enclosure 118 remains engaged to confine fluid escaping from the second end of the lumen 119.

Returning to FIG. 1A, the system 100 can include or be used with a specimen container plug 122 sized and shaped to be removably coupled to the aperture 113 of the specimen container 110. The device 120 can be sized and shaped to replace the specimen container plug 122 during coupling to the specimen container 110. Also, the system 100 can include or be used with a specimen container plug-replacing apparatus for removing a specimen container plug 122 from the aperture 113 of the specimen container 110 and replacing the chamber plug with the device 120. Here, the chamber plug replacing apparatus can be configured to lift, raise, slide, roll, or otherwise move the specimen container plug 122 away from the aperture 113 of the specimen container 110 to help replace of the specimen container plug 122 with the device 120. For example, chamber plug replacing apparatus can concurrently or sequentially remove the specimen container plug 122 from the specimen container 110 and replace the specimen container plug 122 with the device 120. The specimen container plug 122 or the device 120 can be removably coupled to the aperture 113 of the specimen container 110 by a respective coupling member. In an example, such a coupling member can be sized and shaped to be removably coupled to the aperture 113 of the specimen container 110 by a screw thread, a snap-fit fitting, a press fit coupling, a flange, a bayonet fitting, a pin-and-slot coupling, a clamping fitting, or other releasable coupling.

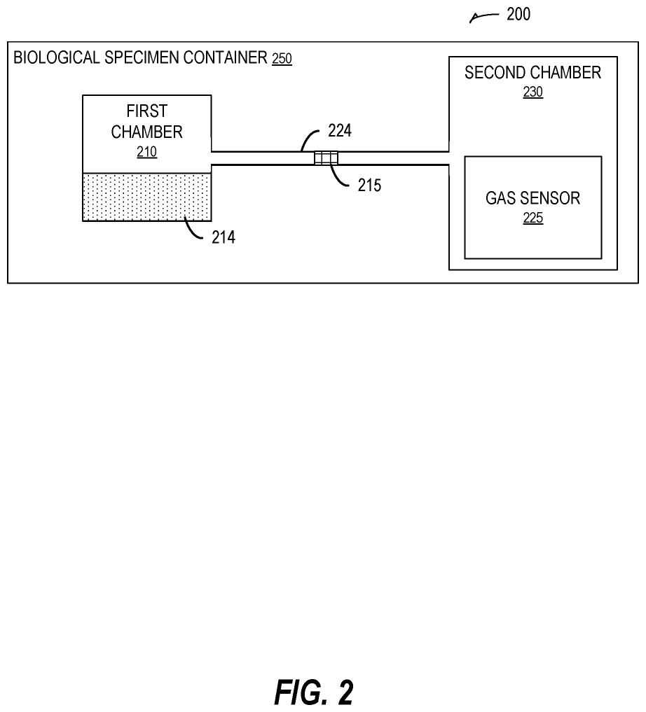

FIG. 2 depicts an example of a gas sensing system. The gas sensing system 200 is similar in many respects to the gas sensing system 100 described above. The gas sensing system 200 can include or use a biological specimen container 250 including a first chamber 210, for containing liquid biological or another specimen sealed therewithin. The biological specimen container 250 can also include a second chamber 230, carrying the gas sensor 225 and a fluid conduit 224 couplable between the first chamber 210 and the second chamber 230. A separator 215 can be included between the first chamber and the second sensor, such as within the fluid conduit 224. In an example, the separator 215 can be manipulated to transition the container 250 from the first state towards a second state, such as to allow gas from the first chamber 220 to diffuse or otherwise flow into the second chamber 230 for sensing by the gas sensor 225 while the biological specimen is located in the first chamber 210. Such a biological specimen container 250 can help enable, e.g., a biological specimen 214 including a liquid component to be prepared, handled, and sensed or imaged later without the risk of damaging the gas sensor or exposing the gas sensor 225 or the specimen 214 to the ambient environment. Separators discussed herein, such as separator 215, can include at least one feature for manipulation of a gas sensing system, such as, e.g., a screw thread mechanism, a threaded shaft mechanism, a pinion gear mechanism, a rack and gear mechanism, or a camming mechanism. Such a mechanism can allow an operator to manually open or close the system via movement or alteration of the separator. Also, one or more locking features can be included, such as to help ensure that the separator remains open, or remains closed, to transition from the first state to the second state, or vice versa. For example, the locking feature can be positioned at an opening to the separator, at a pin that engages the separator, or at a shaft that rotates into engagement with the separator.

The separators discussed herein can also include a plurality of openings (e.g., a plurality of slits, holes, etc.) that can be aligned with each other. The openings can also be configured to cooperate with each other, such as respective openings of the first component and the second component. Also, separators discussed herein may include at least one resilient component, such as an elastomeric material, such as to allow the separator to be manually deformed (e.g., to move) by an operator without requiring a large actuation force.

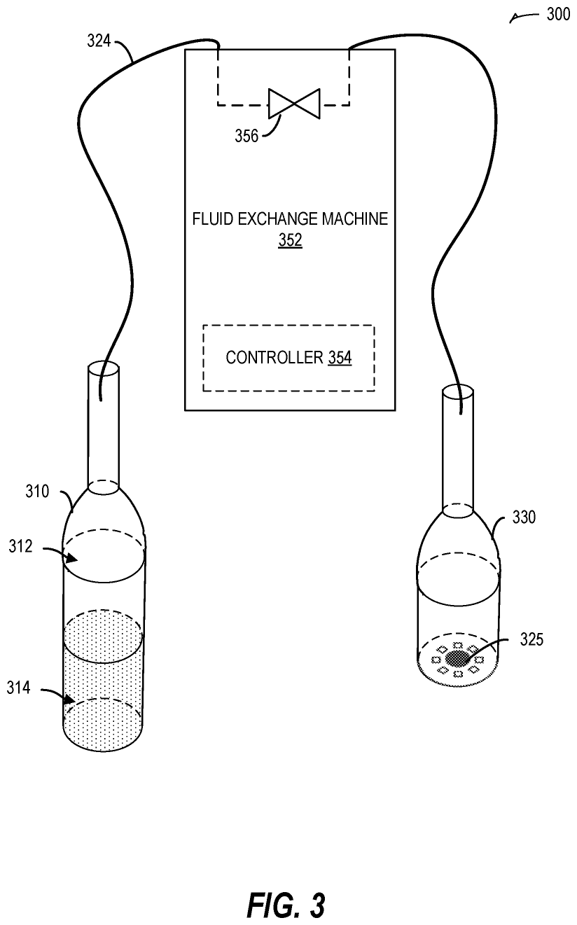

FIG. 3 depicts another example of a gas sensing system. A gas sensing system 300 can include or use a fluid exchange machine 352 to selectively connect a gas headspace 312 between a first container 310 (e.g., a specimen vial) including a biological specimen 314 and a second container 330 (e.g., a gas sensing vial) including a gas sensor 325. Here, the first container 310 and the second container 330 can each be fluidly sealed with respect to the ambient environment. A conduit 324 can be fluidly connected to a peristaltic or other pump of the fluid exchange machine 352 to affect gas exchange from the first container 310 and into the second container 330. In an example, the fluid exchange machine 352 can include a filter or filter assembly to filter particulate matter and bacteria out of the fluid being drawn from the first container 310. The fluid exchange machine 352 can include a mechanism to selectively open a valve connecting the conduit 324 to pump.

Alternatively or additionally, the gas sensing system 300 can include a siphon to supply suction for drawing up at least a liquid portion of the biological specimen 314 into the fluid exchange machine 352 or the second container 330. Here, an inlet of the conduit 324 can be at least partially immersed within the liquid portion of the biological specimen 314 in the first container 310.

A controller 354 (e.g., a microprocessor, microcontroller, or single-chip microcomputer) can be configured to control the operation of the fluid exchange machine 352. The controller 354 can receive an initial signal from a sensor (e.g., the gas sensor 325) to begin a sequence (e.g., start fluid exchange) and can include a signal detector and a timing mechanism to determine the duration of the sequence. A fluid handling assembly, such as a valve assembly 356, can be selectively controlled by the controller to direct the flow of the fluid using the fluid exchange machine 352. For example, a valve assembly 356 can be controlled to open a valve to direct the flow of the fluid from the first container 310 into the second container 330 and can be controlled to close a valve to block the flow of the fluid within the fluid exchange machine 352. Also, a bi-directional valve assembly 356 can be fluidly connected to the conduit 324 and selectively controlled by the controller 354 to allow the flow of the fluid using the fluid exchange machine 352 from the conduit 324 into the second container 330. In this manner, a gas environment associated with the biological specimen 314 within the first container 310 can be drawn into the second container 330. The pressure in the second container 330 can be set by the controller 354 to be the same as the pressure in the first container 310. Also, the controller 354 can control the timing of the draw or the flow rate of the fluid.

A controller 354 (e.g., a microprocessor, microcontroller, or single-chip microcomputer) can be configured to control the operation of the fluid exchange machine 352. The controller 354 can receive an initial signal from a sensor (e.g., the gas sensor 325) to begin a sequence (e.g., start fluid exchange) and can include a signal detector and a timing mechanism to determine the duration of the sequence. A fluid handling assembly, such as a valve assembly 356, can be selectively controlled by the controller to direct the flow of the fluid using the fluid exchange machine 352. For example, a valve assembly 356 can be controlled to open a valve to direct the flow of the fluid from the first container 310 into the second container 330 and can be controlled to close a valve to block the flow of the fluid within the fluid exchange machine 352. Also, a bi-directional valve assembly 356 can be fluidly connected to the conduit 324 and selectively controlled by the controller 354 to allow the flow of the fluid using the fluid exchange machine 352 from the conduit 324 into the second container 330. In this manner, a gas environment associated with the biological specimen 314 within the first container 310 can be drawn into the second container 330. The pressure in the second container 330 can be set by the controller 354 to be the same as the pressure in the first container 310. Also, the controller 354 can control the timing of the draw or the flow rate of the fluid.

In an example, the second container 330 including the gas sensor 325 can be evacuated before being fluidly connected to the gas headspace 312 of the first container 310. For example, the second container 330 can be evacuated to a pressure less than ambient pressure, less than 100 mm Hg, less than 20 mm Hg, or less than 10 mm Hg, less than 5 mm Hg, less than 2 mm Hg, less than 0.5 mm Hg, less than 0.1 mm Hg, less than 0.05 mm Hg, or less than 0.01 mm Hg. The controller 354 can control the duration of an evacuation sequence (e.g., from about 15 seconds to about 2 hours) and/or the rate at which the gas is being drawn from the first container 310.

In an example, a new, unopened, or otherwise clean second container 310 (e.g., the gas sensing vial) can be provided or obtained for testing of an individual biological specimen 314. Also, the second container 310 can be reused for gas sensing of at least two different biological specimens. Here, to avoid residue from a first biological specimen from contaminating a subsequent biological specimen, for example, the second container 310 can be cleaned. Cleaning can involve various methods, such as by using a cleansing agent, such as saline (NaCl), aqueous buffer solutions (e.g., a 5× or 10×TBE), and organic solvents such as methanol, ethanol, ethyl acetate, and chloroform. Other cleaning methods, such as chemical removal (e.g., via acid or base), physical removal (e.g., via laser or heat), electrochemical removal (e.g., via the use of electricity and/or a salt solution), and combinations of these methods can be used. In addition, a cleaning agent can include components such as a biocidal agent, a chelating agent, a protein-binding agent, a surfactant, an enzyme, and/or an antimicrobial agent (e.g., one that is effective against certain bacteria and fungi), or any combination thereof. In an example, the fluid exchange machine 352 can help facilitate such cleaning of the second container 330, such as via an automated process controlled by the controller 354.

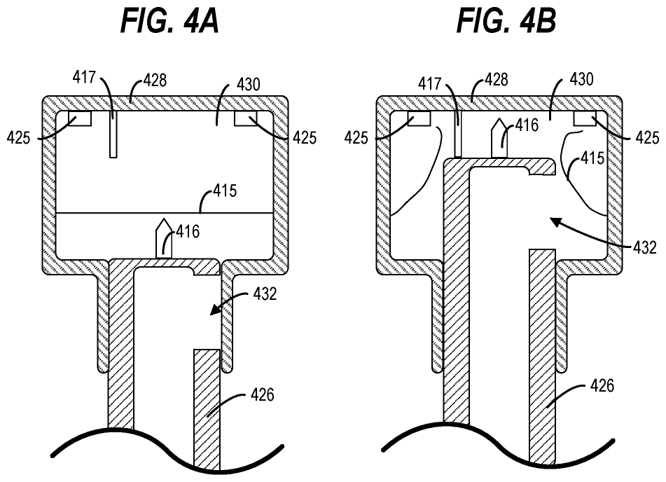

FIG. 4A and FIG. 4B depict an example of a gas sensing system. The gas sensing system 400 can be similar to the gas sensing system 100 described above. Here, the separator can include or use an openable seal 415. In an example, the gas sensing system 400 can include a closable port 432. The gas sensing system 400 can include a first component 426 that can be movable with respect to a second component 428 such as to provide at least one selectively controllable opening between the two that can be used to transition the gas sensing system 400 from a first state and towards a second state. For example, in the first state, a chamber 430 of the second component 428 can be fluidly isolated from the closable port 432 of the first component 426. In the second state, the chamber 430 of the second component 428 can be fluidly connected with the closable port 432 of the first component 426. The first component 426 can be, e.g., coupled to a shaft that can be axially and/or pivotally moved with respect to the second component 428.

As depicted in FIG. 4A, the first component 426 can be at least partially coupled with the second component 428 while maintaining isolation between the chamber 430 and the port 432 in the first state. Here, the port 432 can be closed, such as to be fluidly blocked from the chamber 430 by a wall of the second component 428. Alternatively or additionally, the openable seal can fluidly block the chamber 430 from the port 432, such as regardless of whether the port 432 is closed or open. In an example, the first component 426 can include or use a seal perforator 416. The seal perforator 416 can include, e.g., a sharp point or edge that can puncture the openable seal 415 such as to open a fluid path between the closable port 432 and the chamber 430. Also, at least one of the first component 426 or the second component 428 can include or use a perforator stop 417, such as a standoff, tab, projection, protrusion, notch or ridge, configured to abut the first component 426 or the second component 428 and to prevent the first and second components 426 and 428 from being moved closer towards each other. For example, the perforator stop 417 can help prevent puncturing of the second component 428 by the seal perforator 416. The first component 426 or the second component 428 can also include or be couplable to a source of power, e.g., to supply one or more gas sensing elements 425 with an Electrical current.

For example, when the gas sensing system 400 is moved from a first position (depicted in FIG. 4A) to a second position (depicted in FIG. 4B), the seal perforator can be driven towards the openable seal 415 concurrent with the port 432 being moved to an open position. For example, in moving at least one of the first component 426 and the second component 428 relative to each other towards the second position, the gas sensing system 400 can be moved from the first state towards the second state. Here, fluid can travel between the port 432 and the and the chamber 430, and the port 432 can fluidly connect the chamber to another container including a biological specimen. Thus, the one or more gas sensing elements 425 can become exposed to a gas environment associated with the biological specimen when the system 400 is moved towards the second state.

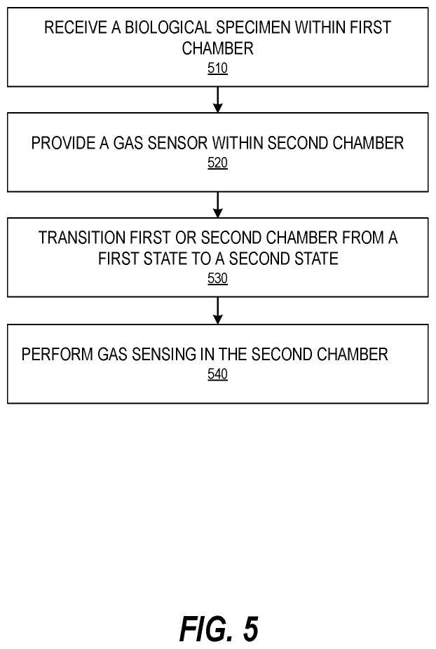

FIG. 5 is a flowchart that describes a method of using a gas sensing system.

At 510, the method can include receiving a biological specimen within a first vessel. At 520, the method can include providing a gas sensor within a second vessel, the gas sensor isolated from the second vessel by an openable seal. At 530, the method can include transitioning from a first state to a second state by opening the openable seal to allow gas from the first vessel to diffuse or otherwise flow into the second vessel for sensing by the gas sensor while the biological specimen can be located in the first vessel. The second vessel can be isolated from a surrounding ambient environment in at least the second state. For example, transitioning can include perforating the openable seal. Transitioning can also include moving a first component of the openable seal with respect to a second component of the openable seal to provide at least one opening in the seal. Further, transitioning can include exchanging the openable seal with a second seal. At 540, gas sensing can be performed, via the gas sensor, while the first and second chamber are transitioned to the second state.

The above Detailed Description can include references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A device for providing access to a biological specimen container, the device comprising:
a fitting sized and shaped to be coupled to a sealed aperture of the specimen container, the fitting including a lumen for allowing fluid communication between first and second ends of the lumen;
a perforator configured to irreversibly sever and unseal an openable fluid-impermeable seal in the sealed aperture of the specimen container via coupling of the fitting to the aperture and to connect the first end of the lumen to a fluid headspace of the specimen container; and
a user-replaceable sealing enclosure sized and shaped to house at least one biological specimen growth indicator at the second end of the lumen, the sealing enclosure selectively user-engageable to confine fluid escaping from the second end of the lumen of the fitting, after the seal has been unsealed by the perforator.

2. The device of claim 1, comprising the at least one biological specimen growth indicator arranged to be enclosed by the sealing enclosure separately from an ambient environment external to the specimen container.

3. The device of claim 2, wherein the at least one biological specimen growth indicator includes a biological culture media arranged to be contacted by fluid from the specimen container via liquid travel from the specimen container through the lumen.

4. The device of claim 2, wherein the at least one biological specimen growth indicator includes a gas sensor including at least one of:
a field effect transistor (FET) including at least one of:
carbon nanotube (CNFET);
silicon carbide (SiCFET);
metal-oxide-silicon (MOSFET);
graphene (GFET);
transition metal dichalcogenide (TMDFET); or
black phosphorus (BPFET);
a camera;
a spectroscopic imaging sensor;
a hyperspectral imaging sensor;
a colorimetric imaging sensor;
a fluorescence response imaging sensor; or
a chromatic sensor or array of chromatic sensors.

5. The device of claim 1, wherein the perforator includes a seal dilator that is sized and shaped to irreversibly sever the openable seal of the specimen container so as to displace greater than 20% of a surface area of the openable seal.

6. The device of claim 1, wherein the sealing enclosure includes a removable cap configured to removably couple with a coupling feature of the fitting.

7. The device of claim 1, wherein the lumen includes a cross-sectional diameter greater than 3 millimeters (mm).

8. The device of claim 1, wherein the device includes an internal fluid sensor for measuring at least one fluid condition within at least one of the lumen or the specimen container.

9. The device of claim 8, wherein the device includes electrical contacts located external to the specimen container and electrically coupled to the internal fluid sensor, the electrical contacts configured to communicate internal fluid sensor data to an external location while the sealing enclosure is engaged.

10. The device of claim 1, wherein at least one of the fitting or the sealing enclosure is formed of at least one of a translucent or transparent material to allow imaging into the specimen container while the sealing enclosure is engaged.

11. A system for gas sensing of a biological specimen, the system comprising:
a first chamber, configured to receive the biological specimen therewithin; and
a gas sensing unit, sized and shaped to be coupled to the first chamber and separated therefrom by an moveable separator to transition from a first state to a second state upon opening the moveable separator to allow entry of gas from the first chamber for sensing by a gas sensor included in the gas sensing unit while the biological specimen is located in the first chamber, wherein the gas sensor is isolated from an ambient environment about the chamber in at least the second state.

12. The system of claim 11, further comprising a chamber plug sized and shaped to be removably coupled to an opening of the first chamber, wherein the gas sensing unit is sized and shaped to replace the chamber plug when the gas sensing unit is coupled to the first chamber.

13. The system of claim 12, in combination with a chamber plug replacer configured to remove a chamber plug from the opening of the first chamber and replace the chamber plug with the gas sensing unit.

14. The system of claim 11, wherein the gas sensing unit comprises:
a second chamber, carrying the gas sensor; and
a fluid conduit couplable between the first chamber and the second chamber and configured to transfer fluid from the first chamber into the second chamber.

15. The system of claim 14, wherein the conduit is included in a siphon to induce fluid from the first chamber into the second chamber.

16. The system of claim 14, wherein the conduit is fluidly couplable to a pump to induce fluid from the first chamber into the second chamber.

17. A method for providing access to a biological specimen container, the method comprising:
coupling a fitting sized and shaped to a sealed aperture of the specimen container, the coupling including providing fluid communication between first and second ends of a lumen of the fitting;
irreversibly severing and unsealing, via a seal perforator attached to the fitting, an openable fluid-impermeable seal in the sealed aperture of the specimen container during the coupling of the fitting to the aperture;
connecting the first end of the lumen to a fluid headspace of the specimen container; and
confining fluid escaping from the second end of the lumen of the fitting, after the seal has been unsealed by the perforator via engaging a user-replaceable sealing enclosure sized and shaped to house at least one biological specimen growth indicator at the second end of the lumen.

18. The method of claim 17, comprising arranging the at least one biological specimen growth indicator to be enclosed by the sealing enclosure separately from an ambient environment external to the specimen container.

19. The method of claim 17, comprising:
providing an internal fluid sensor at a location within at least one of the specimen container or the fitting; and
measuring, via the internal fluid sensor, at least one fluid condition within at least one of the lumen or the specimen container.

20. The method of claim 19, comprising:
providing electrical contacts located external to the specimen container and electrically coupled to the internal fluid sensor; and
communicating internal fluid sensor data, via the contacts, to an external location while the sealing enclosure is engaged.
